(19) United States
(12) Patent Application Publication
(10) Pub. No.: US 2026/0105606 A1
(43) Pub. Date: Apr. 16, 2026

(54) IMAGE PROCESSING APPARATUS, IMAGE PROCESSING SYSTEM, IMAGE PROCESSING METHOD, AND MEDIUM

(71) Applicant: CANON KABUSHIKI KAISHA, Tokyo (JP)

(72) Inventor: YUTA NARUKIYO, Kanagawa (JP)

(21) Appl. No.: 19/352,049

(22) Filed: Oct. 7, 2025

(30) Foreign Application Priority Data
Oct. 10, 2024 (JP) 2024-177952

(51) Int. Cl. G06T 7/00; A61B 6/03; A61B 6/46; G16H 15/00

(52) U.S. Cl. CPC G06T 7/0014; A61B 6/032; A61B 6/461; G06T 2207/10081; G06T 2207/20076; G06T 2207/20081; G06T 2207/20084; G06T 2207/30096; G16H 15/00

(57) ABSTRACT

An image processing method includes steps of: acquiring a medical image; infer a first result relating to a presence probability of a lesion, and a second result relating to a presence region of the lesion; acquiring inference basis information exerting influence on the acquisition of the first result; and determining, based on at least one of the first or second result, as a display target, at least one of the inference basis information or a region based on the second result, determining, as the display target, the region based on the second result when information indicating presence of the lesion is included in the second result, and determining, as the display target, the inference basis information when the information indicating the presence of the lesion is not included in the second result and the information indicating the presence of the lesion is included in the first result.

### BACKGROUND

### Field of the Technology

The present disclosure relates to an image processing apparatus, an image processing system, an image processing method, and a medium.

### Description of the Related Art

There has been known a method of presenting, to a user, a basis of determination in machine learning represented by deep learning. For example, in a method as disclosed in Japanese Patent Laid-Open No. 2024-000600, a task of inferring and classifying a lesion presence probability in an image is executed, a contribution rate of a feature amount in the image that serves as a determination basis therefor is acquired, and the contribution rate is presented to a user. Moreover, in a task of extracting a region of a lesion in an image represented by semantic segmentation and the like, it is also known that the region extracted through machine learning indicates a determination basis.

However, in the case of the method as described in Japanese Patent Laid-Open No. 2024-000600, when it is difficult to visually recognize the lesion in the image in the region extraction task, a failure in region extraction itself may occur, and hence a failure in appropriate presentation of determination basis information may occur.

### SUMMARY

The present disclosure is directed to providing, to a user, suitable determination basis information suggesting presence of a lesion.

According to one aspect of the present disclosure, there is provided an image processing apparatus including: a memory storing instructions; and at least one processor configured to execute the instructions to: acquire a medical image; execute a first task that infers a presence probability of a lesion for the medical image to acquire a first inference result, and execute a second task that extracts a presence region of the lesion for the medical image to acquire a second inference result, the second task being different from the first task; acquire inference basis information being information that has exerted influence at the time of the acquisition of the first inference result; and determine, based on at least one of the first inference result or the second inference result, as a display target, at least one of the inference basis information or a region based on the second inference result, wherein the at least one processor is configured to determine, as the display target, the region based on the second inference result when information indicating presence of the lesion is included in the second inference result, and to determine, as the display target, the inference basis information when the information indicating the presence of the lesion is not included in the second inference result and the information indicating the presence of the lesion is included in the first inference result.

Features of the present disclosure will become apparent from the following description of embodiments with reference to the attached drawings. The following description of embodiments is described by way of example.

### BRIEF DESCRIPTION OF THE DRAWINGS

FIG. 1 is a block diagram for illustrating a configuration example of an image processing system according to a first embodiment.

FIG. 2 is a flowchart for illustrating an example of entire image processing in the first embodiment.

FIG. 3 is a diagram for illustrating an example of entire processing in the first embodiment.

FIG. 4 is a diagram for illustrating an example of an acquisition method for inference basis information in the first embodiment.

FIG. 5A is a view for illustrating an example in which a region extraction result determined by a display target determination unit is superimposed on an input image.

FIG. 5B is a view for illustrating an example in which a presence probability display frame is disposed outside the input image.

FIG. 6 is a block diagram for illustrating a configuration example of an image processing system according to a second embodiment.

FIG. 7 is a flowchart for illustrating an example of entire image processing in the second embodiment.

FIG. 8A is a view for illustrating an example of a generated SPR image.

FIG. 8B is a view for illustrating another example of the generated SPR image.

FIG. 8C is a view for illustrating still another example of the of the generated SPR image.

FIG. 9 is a block diagram for illustrating a configuration example of an image processing system according to a third embodiment.

FIG. 10A is a view for illustrating an example of screen display including a report generation result corresponding to a display target.

FIG. 10B is a view for illustrating another example of the screen display including the report generation result corresponding to the display target.

FIG. 11 is a flowchart for illustrating an example of entire image processing in the third embodiment.

### DESCRIPTION OF THE EMBODIMENTS

Now, an image processing apparatus, an image processing method, and an image processing system being exemplary embodiments for embodying the disclosure of the present specification are described with reference to the drawings. Dimensions, materials, shapes, relative positions of components, and the like described in the following embodiments can be freely set, and can be changed in accordance with a configuration of an apparatus to which the present disclosure is applied and various conditions. The same or equivalent components, members, and pieces of processing illustrated in each drawing are denoted by the same reference symbols in the drawings, and redundant description is appropriately omitted. Moreover, in each drawing, some of components, members, and pieces of processing are appropriately omitted for illustration.

In the following embodiments, the present disclosure is described with reference to, as an example of medical image data, CT image data of images picked up by an X-ray computed tomography apparatus (CT apparatus). However, the present disclosure is not limited to the following exemplary embodiments, and can also be applied to images picked up by, for example, a magnetic resonance imaging (MRI) apparatus, a positron emission tomography (PET) apparatus, and an ultrasonic diagnosis apparatus.

### First Embodiment

In an image processing apparatus according to a first embodiment, region extraction processing of extracting, from an input image input to this image processing apparatus, a region suspicious for a lesion in the image can be executed. In this embodiment, a CT image is used as an example of the input image, and a case in which a pancreatic cancer is a target as the lesion in the image is described. That is, in the image processing apparatus being this embodiment, region extraction processing being processing of extracting a region suspicious for the pancreatic cancer in the CT image is executed.

Subsequently, in the image processing apparatus, region extraction result determination processing of determining whether or not information suggesting the lesion is included in the image as a result of the region extraction processing can be executed. When it is determined that the information suggesting the lesion is included in the image through the region extraction result determination processing, display target determination processing of determining, as a display target to be presented to a user on a display unit such as a monitor, the region extracted through the region extraction processing can be executed.

Moreover, in this image processing apparatus, when it is determined through the region extraction result determination processing that the information suggesting the lesion is not included in the result of the region extraction processing, presence probability inference processing of inferring a probability (for example, likelihood) that the lesion exists in the image can be executed. Then, through use of a result of the presence probability inference processing, it is determined whether or not the lesion is included in the image. As a result, when it is determined that the lesion is included, inference basis information acquisition processing of acquiring information that serves as an inference basis being information which has influenced the presence probability inference processing can be executed. After that, display target determination processing of determining the inference basis information acquired by the inference basis information acquisition processing as a display target to be presented to the user on a monitor or the like can be executed.

In the following embodiments, the pancreatic cancer is given as an example of the target of the processing. However, the portion or the lesion being the target of the present disclosure is not limited to this example, and essential effects of the present disclosure are not lost for other organs and diseases, for example, diseases of various organs such as a liver or a kidney. Moreover, also regarding the lesion, for a lesion other than the cancer given as an example, as long as the region of the lesion can be extracted or the possibility of the presence of the lesion can be known through use of the image, the lesion can be extracted with the present disclosure.

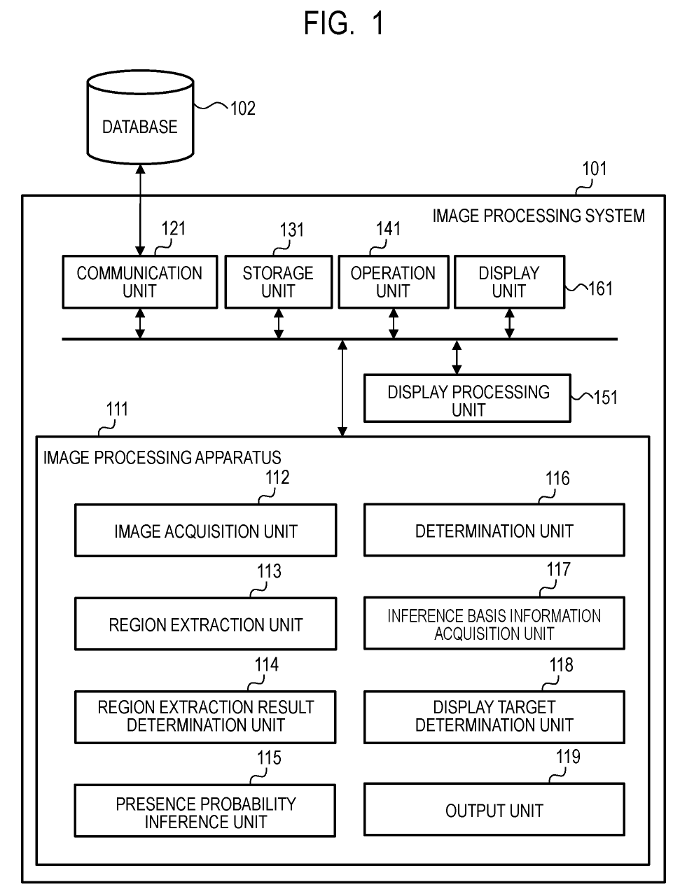

With reference to FIG. 1, configurations of the image processing apparatus and the image processing system according to this embodiment and processing executed in this image processing apparatus are described below. FIG. 1 is a block diagram for illustrating an example of an overall configuration of an image processing system 101 including an image processing apparatus 111 according to this embodiment. As illustrated in FIG. 1, the image processing system 101 includes the image processing apparatus 111 according to this embodiment, a communication unit 121, a storage unit 131, an operation unit 141, a display processing unit 151, and a display unit 161. Moreover, the image processing system 101 can be connected to an external database 102 via the communication unit 121.

The image processing apparatus 111 can be formed of a central processing unit (CPU), or a dedicated or general-purpose processor. The image processing apparatus 111 may be formed of a graphic processing unit (GPU) or a field-programmable gate array (FPGA). Moreover, the image processing apparatus 111 may be formed of an application specific integrated circuit (ASIC), or the like. The image processing apparatus 111 controls each unit in the image processing system 101 to execute processing described below, to thereby implement an image processing method relating to a technology of the present disclosure.

The image processing apparatus 111 according to this embodiment includes an image acquisition unit 112, a region extraction unit 113, a region extraction result determination unit 114, a presence probability inference unit 115, a determination unit 116, an inference basis information acquisition unit 117, a display target determination unit 118, and an output unit 119. Each unit included in the image processing apparatus 111 is described below.

The image acquisition unit 112 is capable of acquiring an inference target image from, for example, the storage unit 131. The inference target image is an image of a target of a subject (patient) acquired through, for example, the CT apparatus. In this embodiment, as an example of the inference target image, it is assumed to acquire a contrast-enhanced CT image of a pancreas parenchymal phase of an abdomen region when approximately 40 to 50 seconds have passed since a contrast agent was injected. However, the contrast phase of the CT image is not limited to the above-mentioned pancreas parenchymal phase, and effects of the present disclosure can be expected for other time phases such as a portal venous phase, an arterial phase, an equilibrium phase, and a late phase. Moreover, a noncontract CT image without the contrast may be used as the inference target image. The image acquisition unit 112 may directly acquire the inference target image from a diagnosis apparatus such as the CT apparatus (not shown), and in this case, the image processing system 101 may be implemented in the diagnosis apparatus as some of functions of the diagnosis apparatus.

The region extraction unit 113 is capable of extracting region information suggesting the pancreatic cancer from the image acquired by the image acquisition unit 112. Processing of extracting the region information may be executed through use of a calculation model implemented in the image processing system 101 or the image processing apparatus 111, or by holding a model structure and parameters of the calculation model in the database 102 or the storage unit 131, and acquiring the model structure and the parameters. The region information is, as an example, such a binary label image that the pancreatic cancer region has 1 and the other region has 0, but the value can freely be set as long as the value varies from region to region. The binary label image is only an example of the region information, and the region information may be in any form as long as the region information indicates the position suggesting the pancreatic cancer. For example, a pancreatic cancer likelihood image holding a value of a likelihood of the pancreatic cancer in each pixel may be used as the region information, or coordinates of a representative position of the pancreatic cancer (for example, coordinates of the center of gravity or coordinates of a rectangle enclosing the pancreatic cancer) may be used as the region information.

The region extraction result determination unit 114 is capable of determining whether or not the pancreatic cancer is included in the image acquired by the image acquisition unit 112 based on the region information suggesting the pancreatic cancer extracted by the region extraction unit 113. Details of processing for the determination to be actually executed are described later.

The presence probability inference unit 115 is capable of inferring the probability value (likelihood) of the presence of the pancreatic cancer from the image acquired by the image acquisition unit 112. The probability value calculated through the inference can be obtained, as an example, such that the value takes 1 when the probability of the presence of the pancreatic cancer is high, takes 0 when the probability of the presence of the pancreatic cancer is low, and takes a continuous value ranging from 0 to 1. Processing of executing this inference may be executed through use of a calculation model implemented in the image processing system 101 as in the region extraction unit 113. As another example, this processing may be executed by holding a model structure and parameters of the calculation model in the database 102 or the storage unit 131, and acquiring the model structure and the parameters.

The determination unit 116 is capable of determining whether or not the pancreatic cancer is present in the image acquired by the image acquisition unit 112 based on an inference result output by the presence probability inference unit 115. Details of processing for the determination to be actually executed are described later.

The inference basis information acquisition unit 117 acquires information on an inference basis being information which has influenced the inference processing by the presence probability inference unit 115 from the image acquired by the image acquisition unit 112 and the inference result output by the presence probability inference unit 115. The information which has influenced the inference processing described here is information suggesting the presence of the pancreatic cancer, and an image feature (image feature amount, image feature value) is given as an example thereof. The image feature in this embodiment is, for example, pancreatic cancer region information in the image, information indicating an obstructed position of a pancreatic duct portion being an indirect finding suggesting the pancreatic cancer, or information indicating atrophy of a part or the whole of the pancreas.

The display target determination unit 118 determines the display target in accordance with, for example, the following condition. When the region extraction result determination unit 114 determines that the pancreatic cancer is included in the region information extracted by the region extraction unit 113 and suggesting the pancreatic cancer, the display target determination unit 118 determines this region information as the display target. Meanwhile, when the region extraction result determination unit 114 determines that the pancreatic cancer is not included in this region information and the determination unit 116 determines that the pancreatic cancer is present, the display target determination unit 118 determines, as the display target, the inference basis information acquired by the inference basis information acquisition unit 117.

The output unit 119 outputs the display target determined by the display target determination unit 118 to the display processing unit 151, and displays the display target on the display unit 161. The determined display target may be output to the database 102 or the storage unit 131 and may be stored therein.

Next, components other than the image processing apparatus 111 which may be included in the image processing system 101 in this embodiment are described. The communication unit 121 is capable of connecting the image processing system 101 to an external apparatus and a network, and can implement communication through predetermined communication means. The communication unit 121 may be formed of, for example, a wireless device such as a Wi-Fi (trademark) device or a Bluetooth (trademark) device or a wired device such as a wired local area network (LAN) or a universal serial bus (USB) device. The communication unit 121 communicates to and from an external database and the like through predetermined communication means, to thereby enable the image processing system 101 to acquire various types of data such as one or more images and the region information.

The storage unit 131 is formed of one or more media which record data, for example, a hard disk drive (HDD) and a random access memory (RAM), and is used for storage of various types of data and storage of various calculation results, for example. The storage unit 131 may be formed of a main storage formed of a volatile memory for temporarily storing read data and the like and an auxiliary storage unit for storing data in a long term as separate components.

The operation unit 141 can be formed of input devices such as a keyboard, a mouse, a touch panel, and a remote controller, and can be used to input an instruction from the user to various devices. Moreover, a mode in which, for example, the operation unit 141 is integrated with the display unit 161 described later and a display screen of the display unit 161 is used as a touch panel can be adopted.

The display processing unit 151 is capable of processing the image and various calculation results output from the image processing apparatus 111 in a form which the display unit 161 can display, and transmitting a processing result to the display unit 161. The display unit 161 is formed of an output device such as a monitor or a display, and can be used to display, to the user, display data such as the calculation results and the various images processed by the display processing unit 151.

In this embodiment, in the database 102 connected to the image processing system 101, for example, the images used in the image processing system 101 are stored. The database 102 is connected to the image processing system 101 via the above-mentioned communication unit 121, and transmits the stored information to the image processing apparatus 111 in response to a request from this image processing system 101. Moreover, the database 102 can store results of the calculation, output images, and the like of the image processing system 101.

In the image processing system 101 illustrated as an example in FIG. 1, the image processing apparatus 111 and the communication unit 121, the storage unit 131, the operation unit 141, the display processing unit 151, and the display unit 161 are illustrated as independent components. However, the image processing system 101 is not limited to the configuration example illustrated in FIG. 1, and the image processing apparatus 111 and some or the whole of the other components may be configured as one unit. Moreover, for example, some or the whole of the components illustrated as being independent of the image processing apparatus 111 may be included in the image processing apparatus 111.

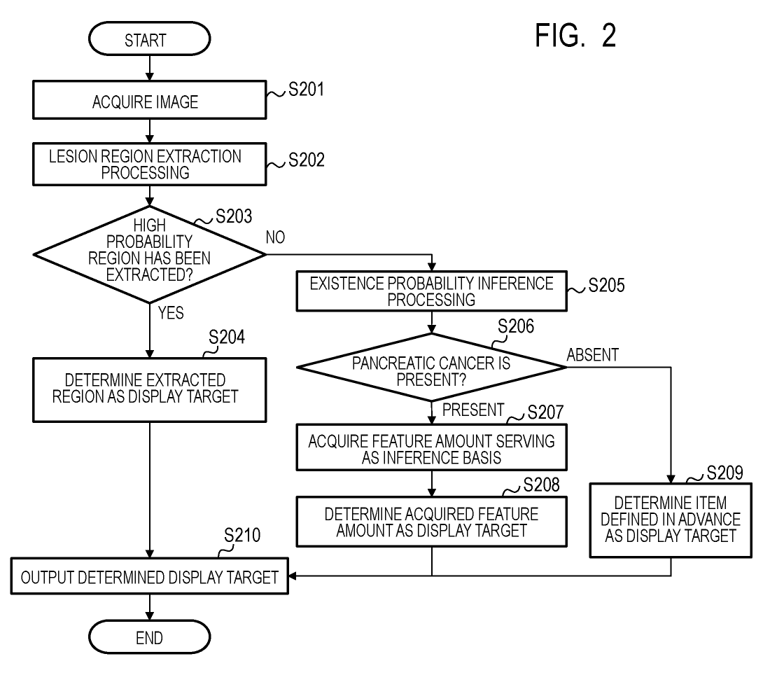

Next, with reference to a flowchart of FIG. 2, an example of image processing actually executed, that is, a series of processing steps up to display of an image, in the above-mentioned image processing system 101 is described in detail. In the description given below, it is assumed that an input image input to the image processing apparatus 111 is stored in the storage unit 131 of the image processing system 101. For example, when an instruction to start the image processing is input by the user through the operation unit 141, the image processing apparatus 111 advances the flow to Step S201 in order to execute the image processing method according to this embodiment.

### Step S201: Acquisition of Image

In Step S201, the image acquisition unit 112 acquires, as the input image, from the storage unit 131, an image specified by the user through use of the operation unit 141. The input image may be acquired by a method other than this method. For example, an image satisfying a predetermined condition may be retrieved from the database 102 and read out to be processed in response to the instruction of the user. Moreover, at this time, the display processing unit 151 may display the acquired input image on the display unit 161. In this embodiment, as the input image, a three-dimensional contrast-enhanced CT image picked up by the CT apparatus is given as an example, but the input image is not limited to this example as described above, and may be an image picked up in another modality, a non-contract CT image, a two-dimensional image, or the like. Moreover, the input image may be a multi-temporal phase image group picked up in a plurality of contrast phases. In this case, a temporal phase that is suitable as the target of Step S202 and the subsequent processing steps may be further selected, or Step S202 and the subsequent processing steps may be executed while each of the images in a plurality of temporal phases is used as the input image. After the acquisition of the image is executed, the image processing apparatus 111 advances the flow to Step S202.

### Step S202: Extraction of Lesion Region

In Step S202, the region extraction unit 113 extracts, as a lesion region, region information suggesting the pancreatic cancer from the input image acquired by the image acquisition unit 112. In this embodiment, it is assumed that, as an example of a method of extracting the region information, semantic segmentation using a convolutional neural network (CNN) being a type of machine learning is used. However, the extraction method for the region information is not limited to this example, and the method may be a neural network method other than the CNN such as a transformer that uses a self-attention mechanism, or a machine learning method other than deep learning such as random forest.

As a form of the region information extracted in this step, for example, such a binary image that the pancreatic cancer region has 1 and the other region has 0 is given as an example. However, the extraction form of the region information is not limited to this example, and the region information may be a likelihood image having, as each pixel value, a continuous value ranging from 0 to 1 indicating the existence likelihood of the region. Moreover, the region information may be a multivalued image in a Gaussian distribution form having a center of the pancreatic cancer as a peak position. At this time, a region information on the pancreas containing the pancreatic cancer may be further acquired, and an extension of the distribution may be changed based on a distance between the center of the pancreatic cancer and a contour of the pancreatic region. Moreover, the region information may be a circumscribed rectangle (3D rectangular parallelepiped) enclosing the pancreatic cancer, or may be, for example, a binary image in which an internal region of the circumscribed rectangle is set to 1 and the other region is set to 0, or may be coordinate values of vertices of the circumscribed rectangle in place of the binary image. The processing of extracting the region information in this step is not processing which extracts the region information in any case. That is, when the pancreatic cancer does not exist in the input image, or when the region information on the pancreatic cancer is not extracted through the extraction processing, there may be a case in which a result that the region information is absent is obtained.

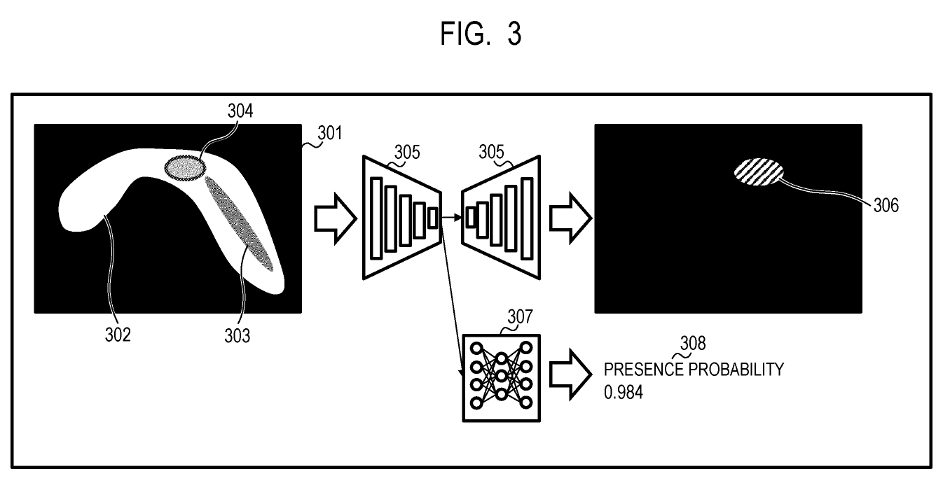

With reference to FIG. 3, details of the region extraction processing by the CNN are now described. FIG. 3 shows a processing example of the segmentation of the pancreatic cancer region by the CNN. In FIG. 3, in an input image 301 being a CT image, a pancreas region 302 and a pancreatic duct region (pancreatic duct 303) are shown. Moreover, the input image 301 includes a pancreatic cancer region 304. In this embodiment, a region extraction unit 305 (corresponding to the region extraction unit 113 of FIG. 1) in this embodiment is formed through use of the CNN, and convolution processing is applied to the input image 301 a plurality of times, to thereby obtain a region extraction result 306.

The region extraction unit 305 in this embodiment is a CNN including an encoder which serves as a feature amount extraction unit and a decoder which restores an image of a desired region mask or the like from the extracted feature amount, and is a CNN, for example, a U-net. The region extraction unit 305 is not limited to the exemplary configuration, and, as another example, may execute only acquisition of the feature extraction result obtained by the encoder, and up-sample the acquired feature amount to transform the feature amount to an input image space, to thereby acquire the region extraction result 306. The feature amount in this embodiment is a result of the convolution processing applied to the image, and hence includes tensor data having a spatial distribution. When the extraction of the region is finished, the image processing apparatus 111 advances the flow to Step S203.

### Step S203: Determination of Region Extraction Result

In Step S203, the region extraction result determination unit 114 determines whether or not the pancreatic cancer is included based on the region information suggesting the pancreatic cancer extracted by the region extraction unit 113. For example, in the case in which the region information is the likelihood image having, as the pixel value, the continuous value ranging from 0 to 1, thresholding of using a predetermined threshold value (for example, 0.5) can be applied to the likelihood image, and such a determination that "the pancreatic cancer is included in the image" can be made when one or more pixels each exceeding the threshold value exists. When the determination that "the pancreatic cancer is included in the image" is made, the condition for the number of pixels each exceeding the threshold value is not limited to the above-mentioned condition of "one or more," and may be freely set, and the condition can be adjusted so that determination processing having high sensitivity or determination processing robust against noise is achieved by changing the condition. In addition, the condition may be set such that the pancreatic cancer is determined to be included when the number of pixels in the maximum connected region of the pixels each exceeding the threshold value (the maximum region in which pixels each exceeding the threshold value are continuous) is equal to or larger than a predetermined number of pixels (for example, 100 pixels). Moreover, the determination may be made based on a volume of the maximum connected region, and, for example, when the volume of the maximum connected region is 100 mm³ or more, the determination that the pancreatic cancer is included may be made. It is only required to calculate the volume of the region by multiplying the number of pixels by a size (volume) of one pixel. The pixel size may be acquired, for example, from the storage unit 131, the database 102, or the like as information accompanying the image, or may be received from the user through the operation unit 141. The threshold value used in the thresholding may be acquired from the storage unit 131, or may be received from the user through the operation unit 141.

Moreover, the region extraction result determination unit 114 can use a method other than the method based on the thresholding to determine whether or not the pancreatic cancer is included. That is, any method can be used as long as the method determines whether or not the pancreatic cancer is included based on the region information extracted by the region extraction unit 113 and suggesting the pancreatic cancer. For example, the determination may be made through a configuration which uses an inference unit based on machine learning which uses this region information as input and outputs whether or not the pancreatic cancer is included.

Moreover, independently of the above-mentioned determination processing, the largest pixel value of the pixel values of the region extraction result may be held as the presence probability of the pancreatic cancer. In this case, in Step S204 described later, the held presence probability can be included in the image determined as the display target as a part of the display target to be displayed on the screen. When it is determined that the pancreatic cancer is included in the image as a result of the above-mentioned determination, the image processing apparatus 111 advances the flow to Step S204. Meanwhile, when it is determined that the pancreatic cancer is not included, the image processing apparatus 111 advances the flow to Step S205.

### Step S204: Determination of Region Extraction Result as Display Target)

In Step S204, the display target determination unit 118 executes processing corresponding to the case in which the region extraction result determination unit 114 determines that the pancreatic cancer is included in the image acquired by the image acquisition unit 112 in Step S203. Specifically, the display target determination unit 118 determines, as the display target, the region information extracted by the region extraction unit 113. After the determination, the image processing apparatus 111 advances the flow to Step S210.

### Step S205: Execution of Presence Probability Inference Processing

In Step S205, the presence probability inference unit 115 infers the probability (presence probability) that the pancreatic cancer is included in the image acquired by the image acquisition unit 112. In this embodiment, the presence probability inference unit 115 outputs, as a result of the inference processing, the probability that the pancreatic cancer is included as a continuous value ranging from 0 to 1. In this case, the presence probability takes 1 or a value close to 1 when the probability of the presence of the pancreatic cancer is high, and takes 0 or a value close to 0 when the probability is low.

Here, as an inference method for the presence probability, for example, a mechanism similar to the encoder of the region extraction unit 113 used in Step S202 can be used. That is, it is possible to execute the processing by the encoder which applies convolution calculation to the input image to calculate a feature amount, and input the calculated feature amount to one of various types of networks for classification such as a fully-connected layer, to thereby output the presence probability of the pancreatic cancer. At this time, as this encoder, the same encoder as the encoder of the region extraction unit 113 used in Step S202 may be used. In this case, the convolution calculation of the input image may be executed in this step, or the calculation result of the encoder being an intermediate result of the processing step of Step S202 may be directly used.

Here, with reference again to FIG. 3, an example of specific processing in this step through use of the CNN is described. The region extraction unit 305 in this embodiment is a CNN, and applies the convolution processing to the input image 301 a plurality of times in the encoder unit to acquire a feature amount map being a result of the convolution. The acquired feature amount map is input to the decoder unit in Step S202, but this step is different therefrom, and the feature amount map is input to the feature amount analysis unit 307 formed of a fully-connected layer or the like in order to analyze the feature amount. The feature amount analysis unit 307 in this embodiment infers a presence probability 308 of the pancreatic cancer based on the feature amount map, but may simultaneously infer, in addition thereto, a presence probability of a tumor which is different from the pancreatic cancer, and is in a difference class (the type of the tumor), for example, a cystic tumor or an intraductal papillary mucinous neoplasm. In this case, the number of output classes of the feature amount analysis unit 307 is changed so that the class to be inferred independently exists for each type of tumor. In FIG. 3, a case in which the presence probability of the pancreatic cancer is inferred as 0.984 is illustrated as an example. When the presence probability is inferred, the image processing apparatus 111 advances the flow to Step S206.

### Step S206: Determination of Presence Probability Inference Result

In Step S206, the determination unit 116 determines, based on the presence probability inferred by the presence probability inference unit 115, whether or not the pancreatic cancer is included in the image acquired by the image acquisition unit 112. Specifically, when the value of the inferred presence probability exceeds a predetermined reference value (for example, 0.5), it is determined that the pancreatic cancer is included in the image. Here, the reference value used for the determination may be acquired from the storage unit 131, or may be received from the user through the operation unit 141. Moreover, in addition to the method based on the comparison with the reference value, any method can be used as long as the method determines whether or not the pancreatic cancer is included from the presence probability inferred by the presence probability inference unit 115. For example, an inference unit based on machine learning which uses the presence probability as input and outputs whether or not the pancreatic cancer is included may be further provided, and the inference unit may be used to make the determination. When it is determined that the pancreatic cancer is included in the image, the image processing apparatus 111 advances the flow to Step S207, and advances the flow to Step S209 otherwise.

### Step S207: Acquisition of Inference Basis Information

In Step S207, the inference basis information acquisition unit 117 acquires the inference basis information being the information which has influenced the inference processing by the presence probability inference unit 115 from the image acquired by the image acquisition unit 112 and the inference result output by the presence probability inference unit 115. Here, the inference basis information in this embodiment is the spatial feature amount contributing to the output of the inference result. Specific examples thereof include an image feature of the pancreatic cancer itself extracted in the image and an image feature indicating an indirect finding suggesting the presence of the pancreatic cancer. Examples of the indirect findings can include a dilated pancreatic duct in which the flow of the pancreatic juice flowing from the pancreatic tail to the pancreatic head is obstructed by ischemic cancer cells, the pancreatic juice is accumulated in the pancreatic duct, and thus the pancreatic duct is dilated, and an obstructed pancreatic duct which is a location of the obstruction of the flow, and other various findings can be expected.

An individual indirect finding is not necessarily be limited to a finding indicating the presence of the pancreatic cancer. However, combination (simultaneous appearance) of detection results of a plurality of indirect findings may be a basis for strong suspicion of the presence of the pancreatic cancer. As an example, even when the obstructed pancreatic duct is confirmed in the CT image, but the pancreatic duct is not dilated and remains thin, a possibility that the pancreatic duct is not obstructed by the cancer cells and the pancreatic duct in a region that is so thin as not to be visually recognizable in the CT image is erroneously recognized as the obstructed pancreatic duct cannot be denied. However, when a finding of the dilated pancreatic duct is simultaneously presented, the possibility of the obstructed pancreatic duct by the cancer cells increases. As described above, it is possible to suspect the presence of the pancreatic cancer by combining the image features indicating the presence of the indirect findings, and hence the image features indicating the indirect findings can be used as the inference basis information,

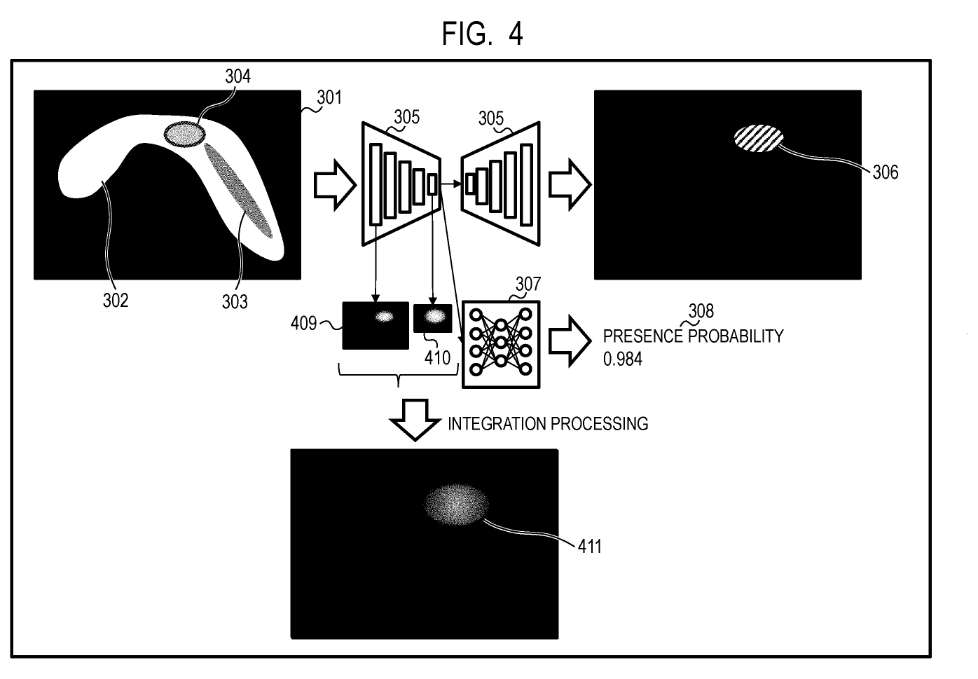

The inference basis information can be acquired through use of a publicly known feature amount analysis method represented by, for example, Grad-CAM as described in a document (Selvaraju, Ramprasaath R., et al. "Grad-cam: Visual explanations from deep networks via gradient-based localization," Proceedings of the IEEE international conference on computer vision, 2017). As an example, with reference to FIG. 4, an acquisition method for the inference basis information is described. The presence probability inference unit 115 (region extraction unit 305 of FIG. 4) in this embodiment is the CNN, and thus applies convolution processing to the input image 301 a plurality of times, to thereby obtain a plurality of feature amount maps 409 being convolution results. The next convolution layer reduces this feature amount map 409, and further applies convolution processing, to thereby acquire the next feature amount map 410. Each of the feature amount maps (409 and 410) obtained in this manner has various values regardless of whether the values are positive or negative. In general, the feature amount maps (409 and 410) indicate various feature amount of the image, and the CNN is trained so that the CNN combines a plurality of the feature amount maps to calculate the feature amount indicating the presence probability of the pancreatic cancer. In FIG. 4, a case in which, as a result of entire execution of the convolution, the inferred presence probability of the pancreatic cancer is 0.984 is illustrated as an example.

Finally, the presence probability inference unit 115 applies integration processing to each of the feature amount maps (409 and 410), to thereby acquire the inference basis information 411. Various methods for the integration processing exist, and, as an example, the inference basis information can be acquired by acquiring a region having a strong output in each of the feature amount maps (409 and 410). When the inference basis information is acquired, not only is the region having a strong output simply selected, but also, as described in the above-mentioned document, the value of the presence probability may be differentiated in each feature amount map, and weighting processing may be applied to an obtained gradient. Moreover, the feature amount map takes both positive values and negative values, but it is desired that only the feature amount map which takes positive values be acquired.

Here, in the case of the CNN having the plurality of convolution layers, when the feature amount map 410 being the convolution result closer to the output layer is selected, an abstract feature of the image is extracted, and hence the inference basis information 411 robust against noise can be acquired. When the resolution of the feature amount maps (409 and 410) is different from the resolution of the input image 301, it is preferred that resolution conversion be applied to the feature amount maps (409 and 410) to match the resolution to the resolution of the input image 301, and then the inference basis information 411 be acquired. Moreover, in addition to the configuration of selecting one of the plurality of feature amount maps (409 and 410), a configuration in which the plurality of feature amount maps (409 and 410) are integrated with one another to acquire the inference basis information 411 may be provided. For example, the feature amount maps (409 and 410) in the respective layers may be converted to maps having the same resolution and then overlapped each other to acquire the maximum value of each layer, to thereby acquire the inference basis information 411. In this case, it is possible to acquire the inference basis information 411 having more accurate position information (coordinate information) for the input image 301. After the feature amount serving as the inference basis is identified, the image processing apparatus 111 advances the flow to Step S208.

### Step S208: Determination of Inference Basis Information as Display Target

In Step S208, the display target determination unit 118 determines, as the display target, the inference basis information extracted by the inference basis information acquisition unit 117. After the determination, the image processing apparatus 111 advances the flow to Step S210.

### Step S209: Determination of Item Defined in advance as Display Target

When the flow is advanced to Step S209, the region extracted as the pancreatic cancer is absent, and further, the feature amount suggesting the presence of the pancreatic cancer is not obtained. Thus, it is preferred that the fact that the probability of the presence of the pancreatic cancer is low be presented to the user. Accordingly, in Step S209, the display target determination unit 118 determines, as the display target, an item defined in advance in order to notify the user that the display target is absent, for example, such an item that "The probability of the presence of the pancreatic cancer is low." As another example, an image having all pixels filled with 0 may be determined as the display target. After the determination of the display target, the image processing apparatus 111 advances the flow to Step S210. In Step S209, instead of determining the display target, cancellation of the display itself may be selected, and the next Step S210 may be skipped.

### Step S210: Output Processing

In Step S210, the output unit 119 outputs the display target determined by the display target determination unit 118 to the display processing unit 151. At this time, the output unit 119 can superimpose the determined display target on, for example, the image (input image 301 illustrated in FIG. 3) acquired in Step S201 to output the resultant image. Moreover, the output result may be stored in the storage unit 131 or the database 102. When the display target is output, the image processing described above is finished.

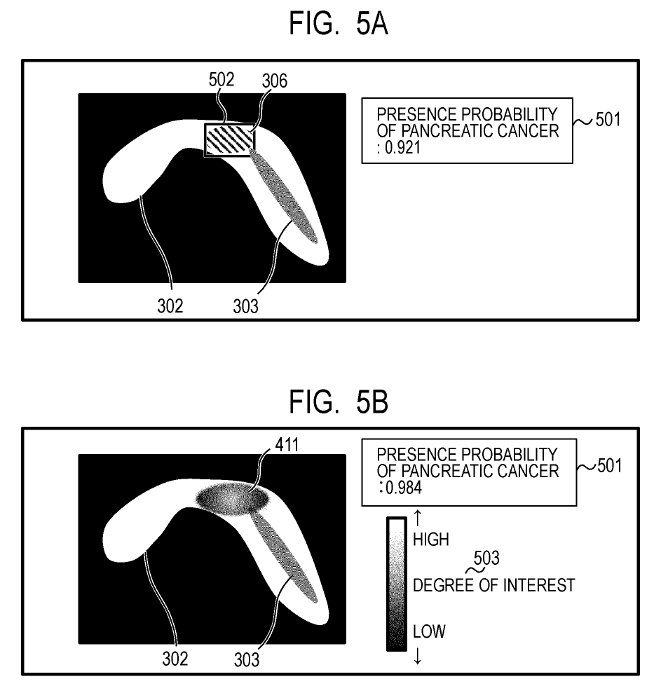

Next, with reference to FIG. 5A and FIG. 5B, an example of the image to be displayed on the display unit 161 by the display processing unit 151 through the above-mentioned image processing is described. FIG. 5A and FIG. 5B are schematic diagrams of images to be displayed on the display unit 161 after the superimposition processing and the like by the output unit 119 in accordance with the display target determined by the display target determination unit 118. In FIG. 5A and FIG. 5B, only an image of a single pancreas and items relating thereto are included in the display example. However, in the actual display, for example, various types of information or images such as information relating to a patient and information on the modality used for the acquisition of this image are also included in the display target, and the various types of information or images can be recognized as at least one of the items determined as the display target.

FIG. 5A shows an example in which the region extraction result 306 determined by the display target determination unit 118 in Step S204 is superimposed on the input image 301 of FIG. 3. The region extraction result 306 output by the region extraction unit 113 exists on the pancreatic cancer region 304, and hence a basis for extraction of the region extraction result is visualized to the user. Moreover, in order to emphasize the region extraction result 306, as an example, a circumscribed rectangle (bounding box) 502 which circumscribes the region extraction result 306 may be displayed. Moreover, when the presence probability of the pancreatic cancer is acquired from the region extraction result 306 in Step S203, a numerical value of the acquired presence probability and the like may be displayed on the screen. As an example, when the maximum pixel value of the region extraction result 306 having a value range of from 0 to 1 is 0.921, it is possible to display the obtained presence probability as 0.921 or 92.1% in a presence probability display frame 501 of the pancreatic cancer. In FIG. 5A, a case in which the presence probability display frame 501 is disposed outside of the input image 301 is illustrated as an example, but the presence probability display frame 501 may be superimposed on the input image 301 for display in association with the region extraction result 306 and the circumscribed rectangle 502.

FIG. 5B shows an example in which the inference basis information 411 determined as the display target by the display target determination unit 118 in Step S208 is superimposed on the input image 301. The inference basis information 411 acquired by the inference basis information acquisition unit 117 indicates the position of the pancreatic cancer (pancreatic cancer region 304) or a position (for example, the obstructed position of the pancreatic duct 303) at which the indirect finding indicating the position of the pancreatic cancer exists, to thereby visualize the inference basis (region of interest) of the presence probability to the user. Here, the inference basis information 411 is information generated from the feature amount map 409 and thus takes both of the positive value and the negative value, and for example, it is possible to report to the user that a region (region in a lighter color of FIG. 5B) having higher pixel values (luminance values) of this feature amount map is a region of more interest. In the example illustrated in FIG. 5B, in order for the user to easily understand a degree of interest of the region to which the displayed inference basis information 411 corresponds, a degree-of-interest scale 503 is displayed. Brightness of the degree-of-interest scale at this time is continuously changed such that, for example, the brightness is close to black when the value of the inference basis information 411 is small, and is close to white when the value is large. The mode of reporting the region of interest to the user is not limited to this example as long as the user can visually recognize this region. Moreover, when the presence probability of the pancreatic cancer is acquired in Step S205, the presence probability of the pancreatic cancer may be displayed on the screen in the same manner as in FIG. 5A.

Through the above-mentioned processing procedure, the processing in the first embodiment of the present disclosure is executed. With this processing, it is possible to provide, to the user, suitable inference basis information suggesting the presence of the lesion.

### Modification Example 1-1

In the above-mentioned first embodiment, the example in which, as the processing steps of Step S202 and Step S205, the region extraction unit 113 and the presence probability inference unit 115 use the common encoder for feature amount extraction has been given for description. However, the embodiment of the present disclosure is not limited to the above-mentioned example. For example, as the feature amount extraction units of the region extraction unit 113 and the presence probability inference unit 115, encoders independent of each other may be used. As an example, as the region extraction unit 113, the CNN having the U-net structure can be used as in the first embodiment, and as the presence probability inference unit 115, a classification model represented by a residual network, VGG, or the like can be used.

As described above, the region extraction unit 113 and the presence probability inference unit 115 include different neural network (NN) models, to thereby be able to acquire different feature amounts between the region extraction task and the presence probability inference task. With this configuration, an effect of increasing variations of the inference basis information to be presented to the user is expected. Moreover, an effect of allowing flexible setting of a learning rate, a model size, and the like appropriate for each task is also expected.

### Second Embodiment

In the first embodiment, the image is used as the input information of each of the region extraction processing and the presence probability inference processing, and the feature amount map being the result of the convolution applied to the image is described as the example of the feature amount having the spatial information. In contrast, an image processing apparatus according to a second embodiment applies visualization processing to the feature amount serving as the inference basis based on the present probability of the lesion inferred through use of a feature amount without spatial information, to thereby execute display target determination processing.

Examples of the feature amount without spatial information in this embodiment include a volume of the pancreas and a representative value (maximum value, minimum value, median, and the like) of the diameter of the pancreatic duct across the entire pancreas. The feature amounts without spatial information are not limited thereto, and for example, a representative value of a diameter of a pancreatic region across the entire pancreas and a radiomics feature amount are also included in the feature amounts without spatial information. In this embodiment, a case in which a representative value of a feature amount calculated from the image such as the radiomics feature amount is used as the input information used for the presence probability inference processing is described. Through use of the representative value of each feature amount, the presence probability inference processing can be executed through simpler processing than in the first embodiment.

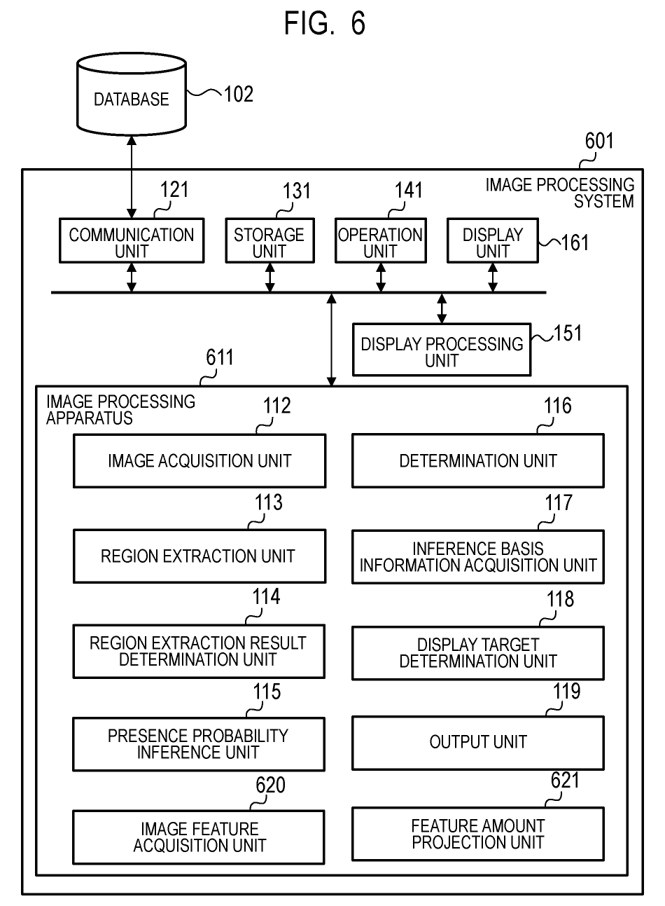

With reference to FIG. 6, configurations of the image processing apparatus and an image processing system according to this embodiment and processing executed in this image processing apparatus are described below. FIG. 6 is a block diagram for illustrating an example of an overall configuration of an image processing system 601 including an image processing apparatus 611 according to this embodiment. As illustrated in FIG. 6, the image processing system 601 further includes, in the image processing apparatus 611, an image feature acquisition unit 620 and a feature amount projection unit 621 in addition to the components of the image processing apparatus 111 described in the first embodiment.

The image feature acquisition unit 620 acquires one or more feature amounts used by the presence probability inference unit 115 from the image acquired by the image acquisition unit 112. The feature amounts acquired in this embodiment are values that do not necessarily include spatial information, for example, the volume of the pancreas and the representative value (maximum value, minimum value, median, and the like) of the diameter of the pancreatic duct.

The feature amount projection unit 621 uses the image feature determined as the inference basis by the inference basis information acquisition unit 117 to project the feature amount to the space of the image acquired by the image acquisition unit 112. That is, the feature amount projection unit 621 projects, to the spatial information, the feature amount serving as the display target used by the output unit 119.

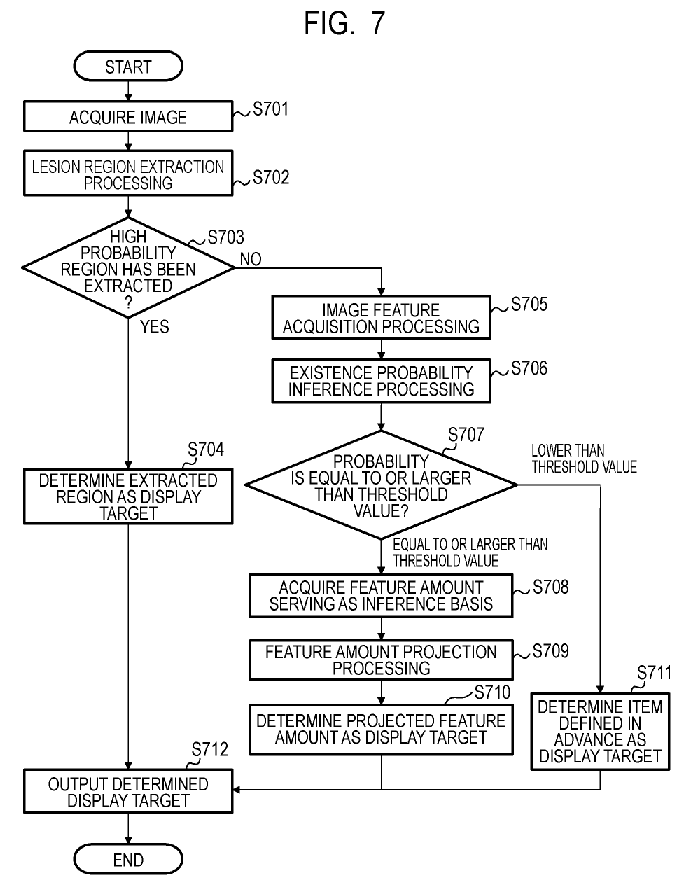

Next, with reference to a flowchart of FIG. 7, an example of image processing actually executed, that is, a series of processing steps up to display of an image, in the above-mentioned image processing system 601 is described in detail. Here, the processing steps of from Step S701 to Step S704 are the same as the processing steps of from Step S201 to Step S204, respectively, of the flowchart of FIG. 2 illustrated as an example in the first embodiment. Moreover, the processing steps of Step S711 and Step S712 are the same as the processing steps of Step S209 and Step S210, respectively. Thus, description of the processing executed in those steps is omitted here, and processing steps different from the processing steps executed in the first embodiment are described. Moreover, in the description given below, it is assumed that the input image is stored in the storage unit 131 of the image processing system 601.

### Step S705: Image Feature Acquisition Processing

In Step S705, the image feature acquisition unit 620 acquires the feature amount used by the presence probability inference unit 115 to infer the presence probability from the image acquired by the image acquisition unit 112. As the feature amount in this embodiment, various feature amounts represented by the radiomics feature amount in the radiation diagnosis field and the like can be used. Specific examples of those feature amounts can include the volume of the target organ (for example, pancreas), shape feature amounts such as a major axis or a minor axis of the target organ in a cross-sectional image, and feature amounts based on a frequency distribution (histogram) of the pixel value and the like. Moreover, in addition thereto, various feature amounts such as a feature amount based on a spatial distribution of the pixel value such as texture can be used. The above-mentioned feature amount may be directly calculated from the input image, or may be calculated from the image obtained by applying spatial conversion to the input image. Moreover, as this feature amount, for example, a feature amount calculated from a converted image after conversion to a stretched curved planar reconstruction (SPR) image generated by virtually stretching, in a straight line form, a pancreatic duct centerline running in the pancreas may be used.

In this embodiment, as an example, a case in which a feature amount relating to the pancreas in an SPR image space is acquired is described. First, pancreatic duct centerline information is acquired based on the input by the user through the operation unit 141. At this time, the pancreatic centerline information may exist in the storage unit 131 or the database 102, or may be acquired from the input image through inference that uses machine learning such as the CNN.

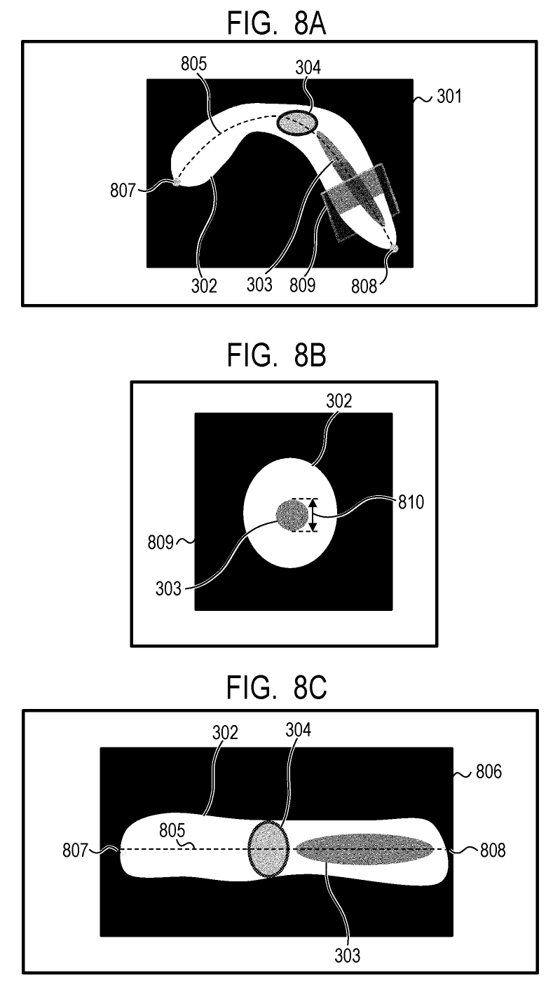

Now, with reference to FIG. 8A to FIG. 8C, a generation example of the SPR image is described. FIG. 8A shows an image obtained through use of the input image 301 illustrated as an example in FIG. 3, and description is given below while the same portions and the like as those of FIG. 3 are denoted by the same reference symbols. The dotted line of FIG. 8A indicates pancreatic duct centerline information 805. The pancreatic duct centerline information 805 is acquired through image processing applied to the input image 301, user input, or the like. Based on the acquired pancreatic duct centerline information 805, points are set, for example, discretely at equal intervals along the centerline from a pancreatic head end 807 to a pancreatic tail end 808. Then, a cross-sectional image 809 is generated by cutting the input image 301 on a cross section passing through each point and orthogonal to a running direction of the pancreatic duct centerline (pancreatic duct centerline information 805). FIG. 8B is a view of the cut cross-sectional image 809 as viewed in the centerline running direction. Further, by layering those generated cross-sectional images 809, an SPR image 806 of FIG. 8C is acquired. FIG. 8C is a view obtained by cutting the SPR image being the layered cross-sectional images along the centerline.

Next, from the obtained SPR image 806, the pancreatic duct diameter being an example of the feature amount used in this embodiment is acquired. The pancreatic duct diameter 810 is acquired as illustrated in FIG. 8B for each generated cross-sectional image from an image end of the SPR image 806 or one end of the pancreatic duct centerline information 805 to the other end thereof, and the acquired value is considered as the feature amount. The feature amount acquired in this step is not limited to the pancreatic duct diameter given as an example, and may be, for example, the pancreas diameter, or a pixel value (luminance value) on the pancreatic duct centerline may be acquired as the feature amount. Moreover, a plurality of feature amounts among those may be acquired. In this embodiment, a case in which the pancreatic duct diameter and the pancreas diameter are acquired is described as an example.

Here, ranges of the acquired values are greatly different from each other between different feature amounts (for example, the pancreatic duct diameter and the pancreas diameter). Thus, normalization processing for roughly unifying distributions of the respective feature amounts is applied so that the values of each feature amount are converted to be included within a range of from 0 to 1. Normalization parameters used at this time may be acquired from the database 102 or the storage unit 131, or may be acquired from the user through the operation unit 141. Moreover, a plurality of images existing in the database 102 or the storage unit 131 may be acquired, and may be normalized through use of the maximum value and the minimum value of each parameter. Each feature amount is divided by a width (range) between the maximum value and the minimum value which the feature amount can take, to thereby normalize the feature amount to the range of from 0 to 1.

Finally, the representative value of each of the acquired feature amounts is acquired. In this embodiment, the maximum value of each feature amount obtained for each of the generated cross-sectional images is used. The representative value is not limited to the maximum value, and may be, for example, a median.

The volume of the pancreas and the diameter of the pancreatic duct may be acquired by, for example, applying a publicly-known image processing method to the input image, or may be acquired from the user through the operation unit 141. As another example, information on the pancreatic duct centerline or a pancreas centerline may be acquired as auxiliary information for acquiring the volume of the pancreas and the diameter of the pancreatic duct, and the volume of the pancreas and the diameter of the pancreatic duct may be acquired based on the acquired information. The information on the pancreatic duct centerline and the pancreas centerline may also be acquired through use of image processing, or may be acquired from the user through the operation unit 141.

### Step S706: Execution of Presence Probability Inference Processing

In Step S706, the presence probability inference unit 115 infers the presence probability of the pancreatic cancer from the representative values of the feature amounts acquired by the image feature acquisition unit 620 in Step S705. As the inference processing, for example, a publicly-known machine learning method of inferring a discrimination problem can be used. Specifically, the representative value of the feature amount can be input to a machine learning model such as a neural network to infer the presence probability. Moreover, logistic regression, a support-vector machine (SVM), or the like may be used. It is assumed that those machine learning models have been trained in advance through use of training data so that the machine learning models can execute appropriate inference. Moreover, the method of acquiring the presence probability is not limited to the method of acquiring the presence probability through the inference that uses a machine learning method, and the presence probability may be acquired through, for example, calculation that uses a mathematical expression having freely-selected coefficients. In this embodiment, through the inference processing, the probability that the pancreatic cancer is included is acquired as the continuous value ranging from 0 to 1. The continuous value takes 1 or a value close to 1 when the probability of the presence of the pancreatic cancer is high and takes 0 or a value close to 0 when the probability is low. When the presence probability is inferred in Step S706, the image processing apparatus 611 advances the flow to Step S707.

### Step S707: Determination of Presence Probability Inference Result

In Step S707, the determination unit 116 determines, based on the presence probability inferred by the presence probability inference unit 115, whether or not the pancreatic cancer is included in the image acquired by the image acquisition unit 112. Specifically, when the value of the inferred presence probability is equal to or larger than a predetermined reference value (for example, 0.5), it may be determined that the pancreatic cancer is included in the image, and otherwise, it may be determined that the pancreatic cancer is not included in the image. When it is determined that the pancreatic cancer is included in the image as a result of the above-mentioned determination, the image processing apparatus 611 advances the flow to Step S708. When it is determined that the pancreatic cancer is not included in the image, the image processing apparatus 611 advances the flow to Step S711.

### Step S708: Acquisition of Inference Basis Information

In Step S708, the inference basis information acquisition unit 117 acquires the inference basis information being the information which has influenced the inference processing by the presence probability inference unit 115 from the image acquired by the image acquisition unit 112 and the inference result output by the presence probability inference unit 115. Examples of the inference basis information in this embodiment include, out of the piece of information formed of the representative values of the plurality of feature amounts, input information that is not excluded but is still held even after thresholding, weighting processing, and the like, and contributes to the increase in presence probability. For example, when a neural network is used as the inference processing by the presence probability inference unit 115, some pieces of information such as the representative value of the pancreas diameter and the representative value of the pancreatic duct diameter are input, and in each layer, multiplication by or addition to other input information is executed. This information which is obtained from the multiplication by or the addition to the other input information, and to which the weighting processing and the bias processing have been applied in an activation function, and has successfully fired a node in the output layer is the inference basis information. In order to obtain this inference basis information, a loss value (1-(inferred presence probability)) obtained through forward propagation is propagated backward, and an obtained gradient can be set as a contribution degree. Then, the feature amount having a large contribution degree can be acquired as the reference basis information. As another example, in a regression analysis method such as the logistic regression, when there exists a feature amount having a large regression coefficient and having a value exceeding a predetermined value, a representative value of the corresponding feature amount can be acquired as the inference basis information. When the inference basis information is acquired, the image processing apparatus 611 advances the flow to Step S709.

### Step S709: Projection Processing for Inference Basis Information

In Step S709, the feature amount projection unit 621 projects the inference basis information obtained in Step S708 to the image space acquired by the image acquisition unit 112. In this embodiment, the representative value of the feature amount selected as the inference basis information is the representative value of the plurality of feature amounts acquired from the plurality of cross-sectional images, and hence coordinate values indicating the position of this representative value in the image space are searched for. In more detail, out of the plurality of feature amounts acquired from the plurality of cross-sectional images, the coordinate values in the SPR space having the same value as the representative value acquired in Step S705 are acquired. At this time, when a plurality of sets of coordinate values in the SPR space having the same value as the representative value exist, the plurality of sets of coordinate values are acquired. Moreover, not only are the coordinate values at the position having the same value as the representative value acquired, but also coordinate values having a feature amount within a predetermined range of difference from the representative value may be acquired. Then, a procedure opposite to the procedure (spatial conversion) of generating the SPR image in Step S705 is applied to the acquired coordinate values to acquire a corresponding position in the image space of the image acquired by the image acquisition unit 112. That is, the coordinate values on the SPR image are projected to the corresponding position on the input image.

Next, the feature amount projection unit 621 generates a display image determined as the display target in Step S712. The feature amount projection unit 621 generates an image having the same shape (pixel arrangement) as the image acquired by the image acquisition unit 112, and initializes pixel values to any value (for example, 0). Then, the feature amount projection unit 621 uses the coordinate values in the image space acquired through the above-mentioned method to draw a likelihood map (heat map) having any size on the display image. Here, the likelihood map is a multivalued image in a Gaussian distribution form having the coordinate values in the image space as a peak position, and the extension of the distribution can be freely set. In this embodiment, the information indicating the region is not limited to the likelihood map, and may take various forms, for example, an arrow pointing the coordinates or a rectangle. By drawing such a likelihood map, an image on which the position serving as the inference basis can be visually recognized can be generated. When the projection processing for the feature amount is finished, the image processing apparatus 611 advances the flow to Step S710.

### Step S710: Determination of Inference Basis Information as Display Target

In Step S710, the display target determination unit 118 determines, as the display target, the information indicating the inference basis position projected by the feature amount projection unit 621. At this time, the image on which the inference basis information is projected becomes the above-mentioned display image determined for Step S712. After the determination, the image processing apparatus 611 advances the flow to Step S712.

Through the above-mentioned processing procedure, the processing in the second embodiment of the present disclosure is executed. With this processing, it is possible to provide, to the user, suitable inference basis information accompanied by spatial information suggesting the presence of the lesion even when the feature amount used for the inference of the presence probability of the lesion is the feature amount without spatial information.

### Modification Example 2-1

In the second embodiment described above, the example in which, as the processing step of Step S705, the image feature acquisition unit 620 acquires, from the SPR image, the representative value of the feature amount such as the diameter of the pancreatic duct to execute the presence probability inference processing has been given for description. However, the embodiment of the present disclosure is not limited to the above-mentioned example. For example, without acquiring the representative value of each feature amount, some or all of the feature amounts acquired on each crosscut on the SPR image can be input to the presence probability inference unit 115 to infer the presence probability of the pancreatic cancer. In this case, the position of each crosscut in the SPR image and the acquired feature amount are associated with each other, to thereby be able to obtain the feature amount having spatial information. As a result, the feature amount projection processing of Step S709 can make projection to the position on the crosscut associated with the feature amount selected as the inference basis information, and hence the processing step of Step S709 can be executed through simpler processing. Thus, an increase in processing speed can be expected.

### Third Embodiment

In the first embodiment, a method for enabling the user to visually recognize to which of the above-mentioned results the display image corresponds by displaying the result of the region extraction processing acquired in Step S202 and the result of the inference basis acquired in Step S207 in different modes has been described. However, the embodiment of the present disclosure is not limited to the above-mentioned example. In an image processing apparatus according to a third embodiment, an example of a method for enabling the user to visually recognize to which of the above-mentioned results the displayed image corresponds by executing report generation in accordance with the display target is described.

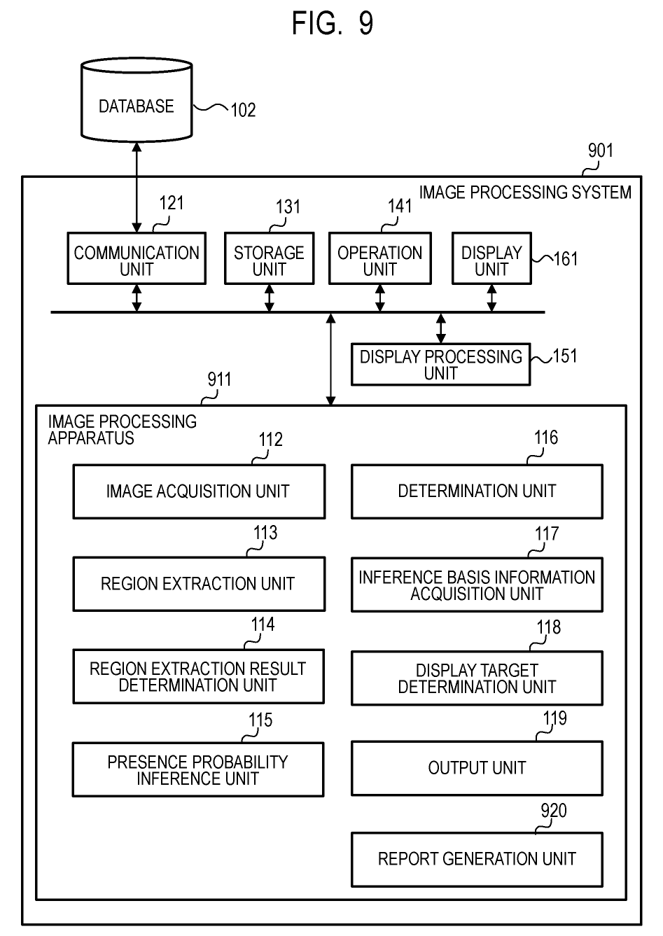

With reference to FIG. 9, configurations of the image processing apparatus and an image processing system according to this embodiment and processing executed in this image processing apparatus are now described. FIG. 9 is a block diagram for illustrating an example of an overall configuration of an image processing system 901 including an image processing apparatus 911 according to this embodiment. As illustrated in FIG. 9, the image processing system 901 further includes, in the image processing apparatus 911, a report generation unit 920 in addition to the components of the image processing apparatus 111 described in the first embodiment.

The report generation unit 920 acquires the information relating to the display target determined by the display target determination unit 118, and generates a report based on this information. The generated report can be included in a display target to be determined, which is described later.

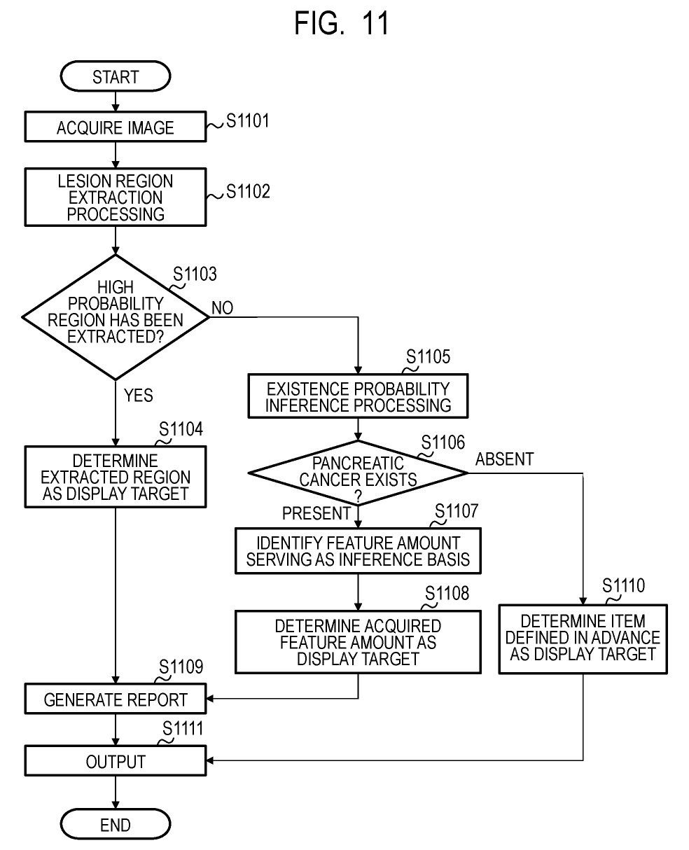

Next, with reference to a flowchart of FIG. 11, an example of image processing actually executed, that is, a series of processing steps up to display of an image, in the above-mentioned image processing system 901 is described in detail. Here, the processing steps of from Step S1101 to Step S1108 are the same as the processing steps of from Step S201 to Step S208, respectively, of the flowchart in the first embodiment. Moreover, the processing steps of Step S1110 and Step S1111 are the same as the processing steps of Step S209 and Step S210, respectively. Thus, description of the processing executed in those steps is omitted here, and processing steps different from the processing steps executed in the first embodiment are described. Moreover, in the description given below, it is assumed that the input image is stored in the storage unit 131 of the image processing system 901.

### Step S1109: Generation of Report

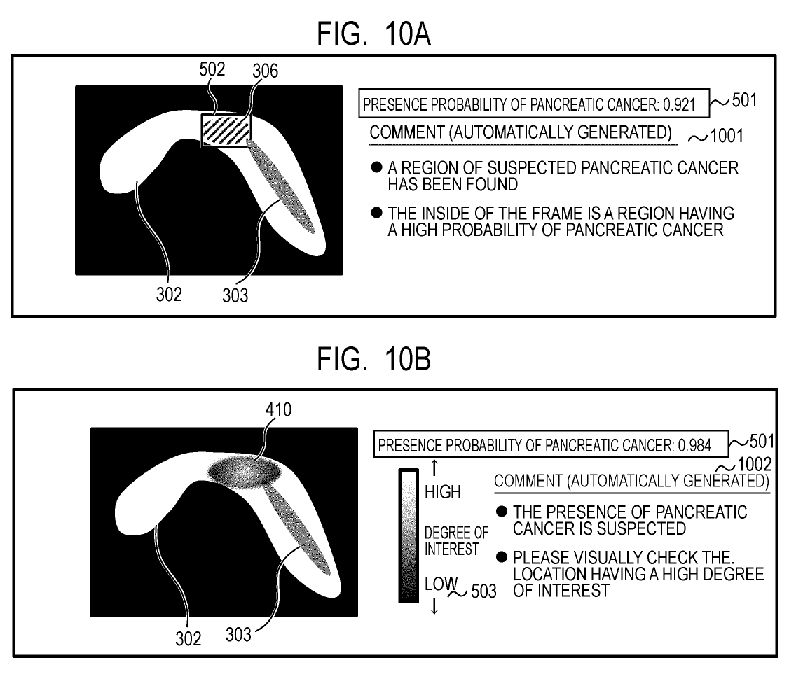

After the image processing apparatus 911 executes the processing steps of from Step S1101 to Step S1108, the report generation unit 920 generates a report in accordance with the display target determined by the display target determination unit 118 in Step S1109. When the display target determination unit 118 determines that the display target is the region output by the region extraction unit 113, the report generation unit 920 generates a report including the fact that the region of suspected pancreatic cancer has been found. As an example, as illustrated in FIG. 10A, the report generation unit 920 generates a report 1001 such as "A region of suspected pancreatic cancer has been found." Moreover, in FIG. 10A, the circumscribed rectangle surrounding the region extraction result generated in Step S209 in the first embodiment is additionally displayed. In this case, an item indicating the emphasized region such as "The inside of the frame is a region having a high probability of pancreatic cancer" may be added to the report 1001. The item written in the report 1001 is an example, and the display item is not limited to this example as long as the display item can present a similar item to the user.

Moreover, when the display target determination unit 118 determines, as the display target, the inference basis information acquired by the inference basis information acquisition unit 117, the report generation unit 920 generates a report including the suspected presence of the pancreatic cancer. As an example, as illustrated in FIG. 10B, the report generation unit 920 generates a report 1002 such as "The presence of pancreatic cancer is suspected." Moreover, in FIG. 10B, the inference basis information is indicated as a continuous value as described in the first embodiment. In this case, regarding the value of the inference basis information as the degree of interest, an item indicating a suspicious region such as "Please visually check the location having a high degree of interest" may be added to the report 1002. In any case, the report generation unit 920 generates a report so as to be different from that in the case in which the display target determination unit 118 determines that the display target is the region output by the region extraction unit 113. The item described in the report 1002 is an example, and the display item is not limited to this example as long as the display item can present a similar item to the user. Through the display of this report in addition to the image and the like, the user who views the content of the report can come to recognize the information determined by the display target determination unit 118 to be displayed. When the report to be displayed is generated, the image processing apparatus 911 advances the flow to Step S1111.

### Step S1111: Output

In Step S1111, the output unit 119 outputs, when the display target determined by the display target determination unit 118 exists, the display target to the display processing unit 151. At this time, the output unit 119 can superimpose the determined display target on, for example, the image (input image 301 illustrated in FIG. 3) acquired in Step S1101, and further add the report generated in Step S1109 thereto to output the resultant image. Moreover, the output result may be stored in the storage unit 131 or the database 102. A case in which the region extracted as the pancreatic cancer is absent and the feature amount suggesting the presence of the pancreatic cancer is not obtained after Step S1103 and Step S1106 can also be expected. In this case, the generation of the report is not required, and thus an item defined in advance is output in Step S1111 after Step S1110.

Through the above-mentioned processing procedure, the processing in the third embodiment of the present disclosure is executed. With this processing, it is possible to suitably provide, to the user through the image and the character information (report sentence), the region extraction result or the inference basis information suggesting the presence of the lesion.

As described above, the image processing apparatus according to one aspect of the present disclosure includes the medical image acquisition unit, the inference unit, the inference basis information acquisition unit, and the display target determination unit. Moreover, the image processing apparatus can also include the output unit 119 which outputs the display target determined by the display target determination unit to the display device, the example of which is the display unit 161. The medical image acquisition unit, the example of which is the image acquisition unit 112 in the first embodiment, can acquire the medical image from the storage unit 131, the database 102, or a medical image pickup device, the example of which is an X-ray CT apparatus (not shown). The inference unit can also include a first inference unit and a second inference unit. In this case, the first inference unit can include the presence probability inference unit 115 and the determination unit 116 given as an example in the first embodiment, and can, as a first task, infer (acquire), as a first inference result, the presence probability of the lesion, the example of which is the pancreatic cancer (Step S205 and Step S206). The second inference unit can include the region extraction unit 113 and the region extraction result determination unit 114, and can, as a second task different from the first task, infer (acquire), as a second inference result, the presence region of the lesion, the example of which is the pancreatic cancer (Step S202 and Step S203). The inference basis information acquisition unit 117 can acquire the inference basis information being the information that has exerted influence at the time of the acquisition of the first inference result by the first inference unit (Step S207). The display target determination unit 118 determines, as the display target, based on at least one (Step S203 and Step S206) of the first inference result or the second inference result, at least one of the inference basis information (Step S208) or the region (Step S204) based on the second inference result. At this time, the display target determination unit 118 determines, when the information indicating the presence of the lesion is included in the second inference result, the region based on the second inference result as the display target (Step S204). Moreover, the display target determination unit 118 determines, as the display target, the inference basis information when the information indicating the presence of the lesion is not included in the second inference result and the information indicating the presence of the lesion is included in the first inference result (Step S208). In the first embodiment, the case in which the image illustrated as an example in FIG. 5A is displayed and the case in which the image illustrated as an example in FIG. 5B is displayed are given as the examples of the case after Step S204 and as the case after Step S208, respectively. However, not only is each of those images displayed separately, but also both of the images may be simultaneously displayed or those images may be displayed in a switchable manner. For example, in the example of FIG. 5B, in a case in which it is difficult to discriminate the image on the superimposed side due to a determined plurality of display targets, for example, in a case in which a plurality of pieces of inference basis information exist, the image of FIG. 5A can easily be checked. Thus, an increase in convenience for the user can be expected. The above-mentioned display of both of the images can be determined under a specific condition, for example, in a case in which a plurality of pieces of inference basis information exist in Step S208 of the flowchart of FIG. 2. As another example, after the display, in the display target output in Step S210, a display form for instruction which enables the switching of the image by the user may be added.

The above-mentioned image processing apparatus can further include an image feature acquisition unit. The image feature acquisition unit acquires, from the medical image, a plurality of image features relating to the organ (pancreas in the embodiment) or the lesion (pancreatic cancer in the embodiment) to be processed. The example of the image feature acquisition unit is given by the inference basis information acquisition unit 117 in the first embodiment, and is given as the configuration further including the image feature acquisition unit 620 in the second embodiment. The above-mentioned inference basis information acquisition unit can acquire the inference basis information based on at least one image feature that has contributed to the first inference result out of the image features. In the first embodiment, the inference basis information acquisition unit can acquire, as the inference basis information, the spatial feature amount in the medical image that has contributed to the inference by the first inference unit.

Moreover, the above-mentioned inference basis information can be acquired as a spatial feature amount formed of the feature amount having the contribution degree calculated from the first inference result, indicating the contribution to the inference by the first inference unit, larger than the predetermined value (Step S207). Moreover, the contribution degree at this time can be based on the gradient acquired based on the inference processing through the first task, and the spatial feature amount at this time can be acquired based on the gradient.

Moreover, the above-mentioned inference unit can execute, when the information indicating the presence of the lesion is not included in the second inference result ("No" in Step S203), the first task for the medical image to acquire the first inference result. Further, the above-mentioned estimation unit can also include the first inference unit which acquires the first inference result and the second inference unit which acquires the second inference result (Modification Example 1-1). The first inference unit and the second inference unit can be formed of the same CNN as described as an example in the first embodiment, but may be formed of different CNNs as described in Modification Example 1-1.

The image processing apparatus according to the present disclosure can further include the feature amount projection unit 621 which projects the image feature to the image space of the medical image as described in the second embodiment. When this configuration is provided, it is preferred that the inference basis information acquisition unit 117 convert, for the image feature to be projected to the image space, a non-spatial feature amount to a spatial feature amount to acquire this feature amount.

The image processing apparatus according to the present disclosure can further include the report generation unit 920 as described in the third embodiment. The report generation unit 920 can generate the sentence suggesting the presence of the lesion when the display target determination unit 118 determines, as the display target, the region based on the second inference result (FIG. 10A). Moreover, the report generation unit 920 can generate the sentence indicating the presence of the region of interest suggesting the lesion when the display target determination unit 118 determines, as the display target, the inference basis information of the first inference result (FIG. 10B).

In the embodiment described above, as the medical image, the X-ray computed tomography (CT) image obtained by the X-ray CT apparatus is given as a preferred example when the present disclosure is to be applied. However, the medical image to which the present disclosure is applied is not limited to the X-ray CT image, and can include medical images obtained in various modalities or under various conditions. Moreover, as the medical image, the image obtained by picking up the abdomen region including the pancreas is given as an example of the preferred image when the present disclosure is to be applied, but the target region is not limited to the pancreas or the abdomen, As long as the portion requires a medical observation in the human body, the portion can be treated as the target region in the present disclosure.

Moreover, the above-mentioned image processing apparatus can further integrally include the display unit 161 separately from the image processing apparatus 111 in the image processing system 101. In this case, this display unit can display the display target determined by the display target determination unit 118. As another example, the image processing apparatus can further integrally include the display processing unit 151 separately from the image processing apparatus 111 in the image processing system 101. In this case, the display processing unit 151 can display, when the display target determination unit 118 determines, as the display target, the region based on the second inference result, the display target on the display unit 161 while the presence region of the lesion is emphasized (FIG. 5A). Moreover, the display processing unit 151 can display, when the display target determination unit 118 determines the inference basis information of the first inference result as the display target, the display target on the display unit 161 while the inference basis information is displayed in a display form different from that of the presence region of the lesion (FIG. 5B). Moreover, the display target determination unit 118 or the output unit 119 may change the display form between the case in which the region based on the second inference result is determined as the display target and the case in which the inference basis information of the first inference result is determined as the display target. For example, by changing a display form such as a display color (lightness of monochrome display or the like), transmittance of the image, or the like, the user can easily determine which of the first inference result and the second inference result is displayed. Moreover, the display processing unit 151 may issue an instruction for such display to the display unit 161 based on the inference result determined as the display target.

Further, the above-mentioned image processing apparatus can include the display unit 161 which displays the display target determined by the display target determination unit 118 separately from those image processing apparatus 111, 611, and 911 to form the image processing systems 101, 601, and 901, respectively. In this case, it is possible to construct a system in which, for example, to each of the image processing apparatus 111, 611, and 911 as a single unit, an individual PC including the display unit 161 is connected, to thereby achieve confirmation of an image processed in each of different examination rooms. Those image processing systems 101, 601, and 901 can also include the display processing unit 151 which displays the display target determined by the display target determination unit 118 on the display unit 161 as a display form in accordance with this display target. Moreover, the image processing systems 101, 601, and 901 can also further include the operation unit 141 which allows input, to the image processing apparatus, of at least one of a threshold value for evaluating the first inference result or a threshold value for evaluating the second inference result when the display target determination unit 118 determines the display target.

According to one aspect of the present disclosure, it is possible to provide, to the user, suitable determination basis information suggesting presence of a lesion.

### Other Embodiment

Embodiment(s) of the present disclosure can also be realized by a computer of a system or apparatus that reads out and executes computer executable instructions (e.g., one or more programs) recorded on a storage medium (which may also be referred to more fully as a 'non-transitory computer-readable storage medium') to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)^{TM}), a flash memory device, a memory card, and the like.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2024-177952, filed October 10, 2024, which is hereby incorporated by reference herein in its entirety.

What is claimed is:

1. An image processing apparatus comprising:
a memory storing instructions; and
at least one processor configured to execute the instructions to:
acquire a medical image;
execute a first task that infers a presence probability of a lesion for the medical image to acquire a first inference result, and execute a second task that extracts a presence region of the lesion for the medical image to acquire a second inference result, the second task being different from the first task;
acquire inference basis information being information that has exerted influence on the first task of the acquisition of the first inference result; and
determine, based on at least one of the first inference result or the second inference result, as a display target, at least one of the inference basis information or a region based on the second inference result,
wherein the at least one processor is configured to determine, as the display target, the region based on the second inference result in a case where information indicating presence of the lesion is included in the second inference result, and to determine, as the display target, the inference basis information in a case where the information indicating the presence of the lesion is not included in the second inference result and the information indicating the presence of the lesion is included in the first inference result.

2. The image processing apparatus according to claim 1,
wherein the at least one processor is further configured to execute the instructions to acquire, from the medical image, a plurality of image features relating to one of an organ or a lesion to be processed, and
wherein the at least one processor is configured to acquire the inference basis information based on at least one image feature that has contributed to the first inference result out of the plurality of image features.

3. The image processing apparatus according to claim 1, wherein the at least one processor is configured to acquire, as the inference basis information, a spatial feature amount in the medical image that has contributed to the first inference result.

4. The image processing apparatus according to claim 3, wherein the spatial feature amount is a feature amount having a contribution degree larger than a predetermined value, the contribution degree being calculated from the first inference result and indicating the contribution to the inference.

5. The image processing apparatus according to claim 4, wherein the contribution degree is based on a gradient acquired based on inference processing through the first task.

6. The image processing apparatus according to claim 5, wherein the spatial feature amount is based on the gradient.

7. The image processing apparatus according to claim 1, wherein the at least one processor is configured to, when the information indicating the presence of the lesion is not included in the second inference result, execute the first task for the medical image to acquire the first inference result.

8. The image processing apparatus according to claim 1, wherein the at least one processor is further configured to execute the instructions to acquire the first inference result and to acquire the second inference result.

9. The image processing apparatus according to claim 2,
wherein the at least one processor is further configured to execute the instructions to project the at least one image feature to an image space of the medical image, and
wherein the at least one processor is configured to acquire, as a spatial feature amount, the at least one image feature projected to the image space.

10. The image processing apparatus according to claim 1, wherein the at least one processor is further configured to execute the instructions to generate a document suggesting the presence of the lesion when the region based on the second inference result is determined as the display target, and to generate a document indicating presence of a region of interest suggesting the lesion when the inference basis information of the first inference result is determined as the display target.

11. The image processing apparatus according to claim 1, wherein the medical image is an X-ray computed tomography (CT) image obtained by an X-ray CT apparatus.

12. The image processing apparatus according to claim 1, wherein the medical image is an image obtained by picking up an abdominal region.

13. The image processing apparatus according to claim 1 further comprising a display to display the determined display target.

14. The image processing apparatus according to claim 1, wherein the at least one processor is further configured to execute the instructions to display the display target such that the presence region of the lesion is emphasized when the region based on the second inference result is determined as the display target, and to display the display target such that the inference basis information is displayed in a display form different from a display form of the presence region of the lesion when the inference basis information of the first inference result is determined as the display target.

15. The image processing system comprising the image processing apparatus according to claim 1 and a display to display the determined display target, wherein the at least one processor is configured to execute the instructions to display the display target such that the presence region of the lesion is emphasized when the region based on the second inference result is determined as the display target, and to display the display target such that the inference basis information is displayed in a display form different from a display form of the presence region of the lesion when the inference basis information of the first inference result is determined as the display target.

16. The image processing system comprising the image processing apparatus according to claim 1, and a display to display the determined display target wherein the at least one processor is configured to execute the instructions to allow input, to the image processing apparatus, of at least one of a threshold value for evaluating the first inference result or a threshold value for evaluating the second inference result when the display target is to be determined.

17. An image processing method comprising:
acquiring a medical image;
executing a second task that extracts a presence region of a lesion for the medical image to acquire a second inference result;
determining, as at least one of display targets, a region based on the second inference result in a case where information indicating presence of the lesion is included in the second inference result;
executing a first task that infers a presence probability of the lesion for the medical image to acquire a first inference result in a case where the information indicating the presence of the lesion is not included in the second inference result, the first task being different from the second task, and acquiring inference basis information being information that has exerted influence on the first task of the acquisition of the first inference result; and
determining, as at least one of the display targets, the inference basis information when the information indicating the presence of the lesion is included in the first inference result.

18. A storage medium having stored thereon a program for causing, when the program is executed by a computer, the computer to execute each step of the image processing method of claim 17.
